SEQUENCE LISTING (22 sequences)

SEQ ID NO: 1 - protein, 32 aa, Homo sapiens
CGNLSTCMLGTYTQDFNKFHTFPQTAIGVGAP

SEQ ID NO: 2 - protein, 32 aa, Mus musculus
CGNLSTCMLGTYTQDLNKFHTFPQTSIGVEAP

SEQ ID NO: 3 - protein, 32 aa, Rattus
CGNLSTCMLGTYTQDLNKFHTFPQTSIGVGAP

SEQ ID NO: 4 - protein, 32 aa, Bos taurus
CSNLSTCVLSAYWKDLNNYHRFSGMGFGPETP

SEQ ID NO: 5 - protein, 32 aa, Canis familiaris
CSNLSTCVLGTYSKDLNNFHTFSGIGFGAETP

SEQ ID NO: 6 - protein, 32 aa, Sus
CSNLSTCVLSAYWRNLNNFHRFSGMGFGPETP

SEQ ID NO: 7 - protein, 32 aa, Ovis
CSNLSTCVLSAYWKDLNNYHRYSGMGFGPETP

SEQ ID NO: 8 - protein, 32 aa, Salmo
CSNLSTCMLGKLSQDLHKLQTFPRTNTGAGVP

SEQ ID NO: 9 - protein, 32 aa, Salmo
CSNLSTCVLGKLSQDLHKLQTFPRTNTGAGVP

SEQ ID NO: 10 - protein, 32 aa, Salmo
CSNLSTCVLGKLSQELHKLQTYPRTNTGSGTP

SEQ ID NO: 11 - protein, 32 aa, Anguilla
CSNLSTCVLGKLSQELHKLQTYPRTDVGAGTP

SEQ ID NO: 12 - protein, 32 aa, Gallus gallus
CASLSTCVLGKLSQELHKLQTYPRTDVGAGTP

SEQ ID NO: 13 - protein, 32 aa, artificial sequence
  Engineered variant of calicitonin
CGNLSTCMLGKLTQELNKLHTFPQTAIGVGAP

SEQ ID NO: 14 - protein, 32 aa, artificial sequence
  Engineered variant of calcitonin
CGNLSTCMLGKLTQELRKLHTFPQTAIGVGAP

SEQ ID NO: 15 - protein, 32 aa, artificial sequence
  Engineered variant of calcitonin
CGNLSTCMLGTYTQDFRKFHTFPQTAIGVGAP

SEQ ID NO: 16 - protein, 32 aa, artificial sequence
  Engineered variant of calcitonin
CGNLSTCMLGKLTQELLKLHTFPQTAIGVGAP

SEQ ID NO: 17 - protein, 32 aa, artificial sequence
  Engineered variant of calcitonin
CGNLSTCMLGKLTQELLKLLTFPQTAIGVGAP

SEQ ID NO: 18 - protein, 32 aa, artificial sequence
  Engineered variant of calcitonin
CGNLSTCMLGKLTQELLKLLTFPRTAIGVGAP

SEQ ID NO: 19 - protein, 52 aa, Homo sapiens
YRQSMNNFQGLRSFGCRFGTCTVQKLAHQIYQFTDKDKDNVAPRSKISPQGY

SEQ ID NO: 20 - protein, 37 aa, Homo sapiens
ACDTATCVTHRLAGLLSRSGGVVKNNFVPTNVGSKAF

SEQ ID NO: 21 - protein, 37 aa, Homo sapiens
ACNTATCVTHRLAGLLSRSGGMVKSNFVPTNVGSKAF

SEQ ID NO: 22 - protein, 37 aa, Homo sapiens [sequence omitted]

(19) United States
(12) Patent Application Publication
(10) Pub. No.: US 2004/0176567 A1
(43) Pub. Date: Sep. 9, 2004

(54) Modified calcitonin

(76) Inventors: Jesus Zurdo, Cambridge (GB); Christopher Martin Dobson, Cambridge (GB)

Correspondence Address: Quarles & Brady, Suite 2040, 411 East Wisconsin Avenue, Milwaukee, WI, 53202-4497, US

(21) Appl. No.: 10/474,635

(22) PCT Filed: Apr. 17, 2002
(86) PCT No.: PCT/GB02/01778

(30) Foreign Application Priority Data
Apr. 17, 2001 (GB) 0109438.2

(51) Int. Cl. C07K 14/585; A61K 38/23

(52) U.S. Cl. 530/307

(57) ABSTRACT

Bioactive peptides are modified to have an increased solubility or reduced tendency to aggregate compared to the wild type modified peptide. In particular modified human calcitonin comprises a peptide having at least 70% identity to SEQ ID NO: 1 and being modified such that the tendency of the modified peptide to aggregate is reduced compared to unmodified human calcitonin.

### FIELD OF THE INVENTION

The present invention relates to modified peptides, having an increased solubility, or reduced tendency to aggregate compared to the wildtype unmodified peptide.

### BACKGROUND OF THE INVENTION

There are numerous bioactive peptides and proteins which are now being proposed for use in the treatment of a variety of conditions. In a number of cases, the use of such peptides has been limited due to the tendency of the peptides to form aggregates following administration to an individual, or when undergoing storage. Such aggregated peptides may be less active than the unaggregated peptide. In some instances, this tendency of the human wildtype peptide or protein to aggregate has lead to the use of peptides from other species which may demonstrate a reduced tendency to aggregate on administration to an individual or during storage. The disadvantage of using such peptides from alternative sources is the possibility of resistance and therefore reduction in the effectiveness of such peptides due to immune responses being generated to the non-human form of the peptides.

An example of such a peptide is calcitonin. Calcitonin causes a rapid but short-lived drop in the level of calcium and phosphate in the blood by promoting the incorporation of these ions in bone. Calcitonin is a 32-residue long polypeptide hormone produced in the C-cells of the thyroid of mammals and it is involved in calcium regulation and bone dynamics (Silverman, S. L., 1997. Am J Med Sci. 313, 13-16).

Salmon calcitonin is currently used instead of human calcitonin as a drug to treat osteoporosis and Paget's Disease. The reason is that human calcitonin has a high tendency to aggregate both in vivo and in vitro even at low concentrations (Sletten, K. et al., 1976, J Exp Med. 143, 993-998; Silver, M. M. et al., 1988, J. Histochem Cytochem. 36, 1031-1036; Siebel, Petal., 1970, Helv Chim Acta. 53, 2135-2150; Arvinte, T. et al., 1993, J Biol Chem. 268, 6415-6422). This constitutes a serious problem during the production, processing and administration of the drug, since aggregated calcitonin is not able to exert its physiological function and can potentially generate an undesired immune response or generate cytotoxicity, as reported for other therapeutic polypeptides when are aggregated (Braun, A. et al., 1997, Pharm Res 14, 1472-8; Curatolo, L. et al., 1997, Cytokine 9,734-9; Brange, J. et al, 1997, J Pharm Sci 86, 517-25).

Salmon calcitonin however is less prone to aggregation, although has a lower activity than the human variant (when aggregation of the latter is prevented) (Cudd, A. et al., 1995, J Pharm Sci. 84, 717-719). Salmon calcitonin bears a low sequence identity with the human variant (16 differences in 32 residues or 50% sequence identity). For this reason, long term treatment with the salmon variant is susceptible to generate resistance or allergy due to the generation by the patient of antibodies against the drug (Levy, F. et al., 1988, J Clin Endocrinol & Metabol. 67, 541-545; Muff, R. et al., 1991, Osteoporos Int. 1, 72-75; Grauer, A. et al., 1995, Exp Clin Endocrinol Diabets. 103, 345-351). When this occurs, it is necessary to increase the dose of drug administered and eventually the treatment can be yielded totally ineffective.

### SUMMARY OF THE INVENTION

The present invention seeks to design modified forms of wild type bioactive peptides in which the solubility of the peptide is increased, or the tendency to aggregate is reduced. Such bioactive peptides show higher sequence identity to the human form of the peptide when compared to Salmon calcitonin yet demonstrate a lower tendency to aggregate or are more soluble than that human form. The provision of such peptides having a low aggregation tendency compared to the wild type sequence allows for easier production and manipulation of the peptides. In addition, when aggregation is prevented, lower doses of the drug can be used to achieve the same effect, therefore reducing the probability of generation of resistance to the peptides by the patient. Moreover, by retaining higher sequence identity with the human variant, allows for the modified peptides to have a physiological activity more similar to that of the human sequence, and therefore minimise the manifestation of side effects and undesired responses.

In accordance with the present invention, we provide modified human calcitonin, comprising a peptide having at least 70% identity to SEQ ID No 1 and being modified such that the tendency of the modified peptide to aggregate is reduced compared to unmodified human calcitonin.

The modifications described herein for calcitonin can also be applied to related peptides, such as calcitonin related peptide I, and other bio-active peptides and proteins such as IAPP. Thus, in another aspect the invention provides, a modified bioactive peptide, having at least 70% identity to a naturally occurring bioactive peptide, wherein the peptide is modified to reduce the tendency to aggregate, or increase the solubility compared to unmodified peptide. Preferably the modifications are selected such that amino acid residues that are polymorphic between species are selected for mutation.

### DESCRIPTION OF THE FIGURES

FIG. 1 compares the turbidity of wildtype human calcitonin to a modified calcitonin in accordance with the invention to provide an indication of the extent of aggregation of the peptides.

FIG. 2. Secondary structure of calcitonin peptides measured by circular dichroism. Experiments reflect the thermal denaturation profile of the peptides at pH 7.0 and 3.0 respectively. Ellipticity was measured at 222 nm and reflects the α-helical content of the peptides. Helical content based on CD spectra calculations (25° C.) according to Chen et al. (1974). Biochemistry 13, 3350-59.

FIG. 3 shows the effect of different calcitonin peptides on the intra-cellular cAMP levels of T47D cells.

FIG. 4 shows intrinsic a-helical propensities calculated for several human calcitonin-related peptides (CGRP-1, CGRP-2, IAPP and Adrenomedullin). Predictions were made using the predictive algorithm Agadir. Similar profiles were obtained using the PDH and GOR-4 algorithms. The graphs represent the predicted percentage of a single residue to populate a α-helical conformation. Regions with higher values represent the preferred areas for engineering peptide variants to have enhanced α-helical content.

### DESCRIPTION OF THE SEQUENCES

SEQ ID NO: 1 is human calcitonin having the sequence CGNLSTCMLGTYTQDFNKFHTFPQTAIGVGAP.

SEQ ID NO: 2 is the amino acid sequence for mouse calcitonin.

SEQ ID NO: 3 is the amino acid sequence for rat calcitonin.

SEQ ID NO: 4 is the amino acid sequence for bovine calcitonin.

SEQ ID NO 5: is the amino acid sequence of dog calcitonin.

SEQ ID NO: 6 is the amino acid sequence for pig calcitonin.

SEQ ID NO: 7 is the amino acid sequence for sheep calcitonin.

SEQ ID NO: 8 is the amino acid sequence for salmon-3 calcitonin.

SEQ ID NO: 9 is the amino acid sequence for salmon-2 calcitonin.

SEQ ID NO: 10 is the amino acid sequence for salmon-1 calcitonin.

SEQ ID NO: 11 is the amino acid sequence for eel calcitonin.

SEQ ID NO: 12 is the amino acid sequence for chicken calcitonin.

SEQ ID NOs: 13-18 are modified polypeptides according to the invention.

SEQ ID NO: 19 is the amino acid sequence of human adrenomedullin.

SEQ ID NO: 20 is the amino acid sequence of human calcitonin gene-related peptide-1.

SEQ ID NO: 21 is the amino acid sequence of human calcitonin gene-related peptide-2.

SEQ ID NO: 22 is the amino acid sequence of human LAPP (amylin)

### DETAILED DESCRIPTION OF THE INVENTION

The invention provides a modified bioactive peptide, having at least 70% identity to a naturally occurring bioactive peptide, wherein the peptide is modified to reduce the tendency to aggregate, or increase the solubility compared to unmodified peptide. In one aspect the invention provides modified human calcitonin, comprising a peptide having at least 70% identity to SEQ ID No 1 and being modified such that the tendency of the modified peptide to aggregate is reduced compared to unmodified human calcitonin.

A bioactive peptide for modification in accordance with the present invention is any peptide which has utility in therapy or diagnosis. In particular, peptides whose usefulness is limited by the tendency to aggregate are encompassed by the present invention. Calcitonin is an example of such a peptide. Bioactive peptides for modification in accordance with the present invention are preferably human peptides, for administration to humans in need of treatment thereof.

In accordance with the invention such a bioactive peptide is modified to reduce the tendency of the peptide to aggregate, or to increase the solubility of the peptide. Preferably, a modified peptide retains at least 70% identity to an unmodified peptide, preferable at least 80%, 84%, 87%, up to 90% identity with the unmodified peptide. Preferably, the modifications to the peptides of the invention are done by substitution. Thus, % identity may calculated by obtaining the best possible alignment with an unmodified peptide, without including gaps in the sequences and calculating the number of amino acid substitutions compared to the wild type sequence.

In accordance with the present invention, a modified polypeptide may have 1, 2, 4, 5, 6, 7, 8, 9 up to 10, 15, 17, 20, 30 or 40 amino acid modifications, with fewer modifications such as 2, 4, 5, 6, 7,or 8 preferred, particularly for shorter peptides such as those of 20, 30 or 40 amino acid residues in length. For example, in the case of calcitonin, a 32 amino acid peptide, the peptide preferably has no more than 8, no more than 7 amino acid changes compared to SEQ ID No 1 and most preferably 6 or 5 or fewer modified amino acids compared to the wild type sequence. Preferably the modifications are kept to a minimum, such that a modified peptide retains some if not all of the activity of the unmodified peptide.

Preferred amino acids for modification can be identified in a number of ways. Modifications are preferably made outside of the active site of a peptide. Preferred regions for modification include those for which the peptide is polymorphic amongst different species. For example, for calcitonin, the human form may be aligned with the salmon form of calcitonin. Polymorphic residues can be identified and targeted for potential modification in the-human form of calcitonin. Modifications may be made such that an amino acid in a human form of the bio-active peptide is modified to that which is present at that position in the peptide from another species.

Preferably, amino acid replacements are selected that increase the propensity of the polypeptides to form local interactions of the α-helical type. Helical propensities for the different polypeptides can be predicted using the semi-empirical agadir algorithm (Muñoz and Serrano, 1994, Nature Struct Biol 1, 399-409; Muñoz & Serrano, 1994, J Mol Biol 245, 297-308; Muñoz & Serrano, 1997 Biopolymers 41, 495-509 and Lacroix et al 1998, J Mol Biol 284, 173-191), as well as other available algorithms including PHD (Rost, B. et al, 1993, J. Mol. Biol., 232, 584-599), PROF (Rost, B. et al, 1996, Methods Enzymol. 266, 525-539) and GOR4 (Garnier J et al, 1978, J Mol Biol, 120, 97-120; Garnier J et al, 1996, Methods Enzymol, 266, 540-553). Additional algorithms based on structural databases, structural preferences databases and rotamer preference databases can be also used and or designed for this purpose.

Peptide modifications are carried out such that the number of hydrophobic residues is reduced or to increase the net charge of the peptide. Thus, where a human form of the bio-active peptide includes a hydrophobic amino acid, and a non-human form has a polar or less-hydrophobic residue at the same position, such a residue is selected for modification either to the amino acid residue in the non-human form, or to another polar or less hydrophobic residue. Amino acid hydrophobicity can be established using any of the described scales (Kyte J., Doolittle R. F., 1982, J. Mol. Biol. 157:105-132; Black, S. D. etal., 1991, Anal. Biochem. 193, 72-82; Wimley, W, C, & White, S. H., 1996, Nature Struct. Biol. 3, 842-848; Wimley, W. C. etal, (1996). Biochemistry 35, 5109-5124). Additionally, the introduction of amino acid substitutions to increase the net charge assists in the designed sequence by generating repulsion between different peptide chains and thus contributes towards increasing the solubility of the peptide and decreasing its aggregation. The introduction of basic residues may be particularly preferred since the charge effect of such a substitution would also operate at low pH values. We set out in the table below conserved amino acid substitutions.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Where it is desired to increase the net charge, such substitutions may be non-conservative but are selected to have the desired effect of increasing charge, or reducing hydrophobicity of the polypeptide.

The invention is hereinafter described in more detail by way of example only with particular reference to calcitonin. Calcitonin is a 32 amino acid peptide. Comparisons were made with calcitonin from other species to identify suitable amino acids for modification. Modifications to increase helical interactions are located preferentially in those areas of the peptide with higher helical propensity. Preferably α-helical propensities for each residue are estimated using predictive algorithms, such as Agadir, PDF, PROF or GOR-4 or any other algorithm based on protein and peptide structural databases. In a preferred aspect of the invention, amino acid changes are selected to occur outside of the active site. In particular, the modifications are selected such that they do not affect the cyclic N-terminus domain in which cysteines at positions 1 and 7 form a disulphide bridge. In one aspect of the present invention, the alterations or substitutions lie within the middle section of the peptide, which is also predicted to be the more α-helical. It is particularly preferred that substitutions are not made within the N-terminus, for example within the 5 N-terminal amino acids, preferably not within the 10 N-terminal amino acids. Alternatively or additionally, preferably, modifications are not made within the 5 C-terminal amino acids and preferably not within the 10 terminal amino acids. Preferred modifications are listed below, using the 1 letter amino acid code and based on the N-terminal amino acid of human calcitonin being numbered residue 1: T at position 11, Y at 12, D at 15, F at 16, N at 17, F at 19, H at 20 and Q at 24.

Preferred substitutions are those which reduce the hydrophobic nature of the amino acid residue, or decrease the tendency of the peptide to aggregate. Preferred substitutions are those which incorporate an amino acid residue which is found at the equivalent position in calcitonin from another species, or adopt the same charge as those found in other species. Particularly preferred substitutions include T to K at 11, Y to L at 12, D to E at 15, F to L at 16, N to L or R at 17, F to L at 19, H to L at 20 and Q to R at 24.

In a particularly preferred aspect of the invention, the polypeptide comprises SEQ ID NO: 1 having modifications at one or more of positions 11, 12, 15, 16, 17, 19, 20 and 24. In a particularly preferred aspect of the invention, the polypeptide comprises at least two modifications at these positions. In preferred aspects of the invention, the variant calcitonin will not incorporate any additional modifications other than modifications at positions 11, 12, 15, 16, 17, 19, 20 or 24. In preferred aspects of the invention, the modified polypeptide is not SEQ ID NO: 2 or SEQ ID NO: 3. The polypeptides of the invention may be modified, for example by the addition of histidine residues to assist their identification or purification. Such additional residues may be cleaved prior to use. Alternatively, signal sequences may be added to promote their secretion from the cell where the polypeptides are to be produced recombinantly. Other conventional modifications may be included such as cyclic N-terminus and/or C-terminus amidation.

A modified peptide in accordance with the invention has a reduced tendency to aggregate or an increased solubility compared to the unmodified peptide. Preferably, the modified peptide will retain some or all of the activity of the unmodified peptide. Such activity can be readily monitored for example by in vitro cellular assays or using an appropriate animal model depending on the peptide involved.

Aggregation properties can be monitored by any suitable method. Modified peptides can be designed and their aggregation properties monitored by computer modelling. Alternatively studies may be carried out, for example by incubating the peptide in vitro. The turbidity of the incubated peptide solution may be monitored over a period of hours days or weeks. Comparison with turbidity measurements for a unmodified peptide may also be taken as a control to assess the affect of the modifications on the aggregation of solubility properties of the peptide.

Polypeptides designed to encompass modifications as defined above can be produced by any method of polypeptide synthesis known in the art. Typically polypeptides of the invention are produced by chemical synthesis or recombinant in vitro or in vivo expression.

Polypeptides may be chemically synthesised using various solid-phase techniques (e.g. Roberge et al. 1995) and automated synthesis may be achieved, for example, using the ABI 431 A Peptide Synthesizer (Perlcin Elmer).

Recombinant in vivo or in vitro production of polypeptides can be achieved by the expression of a polynucleotide that comprises a sequence that encodes a polypeptide of the invention.

A further embodiment of the invention provides a polynucleotide which comprises a sequence that encodes a polypeptide of the invention. The polynucleotide sequence may be designed with reference to the degeneracy of the genetic code and in light of the preferred codon-usage for any particular organism in which the polynucleotide might be expressed. The polynucleotide may be DNA or RNA, and may be single or double stranded, that is comprising a polynucleotide of the invention and its complement. They thus consist essentially of DNA or RNA encoding the amino acid sequence of the invention.

The polynucleotides may include within them synthetic or modified nucleotides. A number of different types of modification to polynucleotides are known in the art. For the purposes of the present invention, it is to be understood that the polynucleotides described herein may be modified by any method available in the art. Such modifications may be carried out, for example, in order to change the in vivo activity or lifespan of polynucleotides of the invention.

Polynucleotides of the invention may be used to produce a primer, e.g. for use in PCR (polymerase chain reaction), or alternative amplification reaction (for example to facilitate amplification or site directed mutagenesis). Such primers and other fragments will be at least 15, preferably at least 20, for example at least 25, 30 or 40 nucleotides in length, and are also encompassed by the term polynucleotides of the invention as used herein.

Polynucleotides such as a DNA polynucleotide and primers according to the invention and the unmodified forms thereof may be produced recombinantly, synthetically, or by any means available to those of skill in the art. They may also be cloned by standard techniques. Thus polynucleotides may be cloned into any vector available in the art. The polynucleotides are typically provided in isolated and/or purified form.

In general, short polynucleotides of the invention, e.g. primers, will be produced by synthetic means, involving a step wise manufacture of the desired nucleic acid sequence one nucleotide at a time. Techniques for accomplishing this, using automated techniques, are readily available in the art.

Longer polynucleotides of the invention and of unmodified forms may be produced by combining short polynucleotides using standard techniques, for example by ligation. They may also be produced by recombinant means, for example using PCR cloning techniques. This will involve making a pair of primers (e.g. of about 15-30 nucleotides) to a region of the gene which it is desired to clone and bringing the primers into contact with a target polynucleotide. For the unmodified form, the target polynucleotide used is typically obtained from a cell in the form of genomic DNA (to allow cloning of the whole gene, typically including introns and promoter regions) or mRNA, or cDNA prepared therefrom. For production of polynucleotides of the invention, small quantities of the polynucleotide, produced by any means, may be used as the target polynucleotide in a PCR amplification reaction. Amplification is performed under suitable conditions to bring about selective amplification. Following amplification of the desired region, the amplified fragment may be isolating (e.g. by purifying the reaction mixture on an agarose gel) and recovered. The primers may be designed to contain suitable restriction enzyme recognition sites so that the amplified DNA can be cloned into a suitable cloning vector.

Although in general the techniques mentioned herein are well known in the art, reference may be made in particular to Sambrook & Russell (Sambrook & Russell, Molecular Cloning, a laboratory manual, 3^{rd} edition. 2001, Cold Spring Harbor Laboratory Press, New York and earlier editions e.g. 1989).

Polynucleotides of the invention may be obtained by site directed mutagenesis of a polynucleotide comprising the unmodified sequence. This technique may be performed in any manner. Typically the technique may be performed by PCR. Such techniques typically include the use of a primer that comprises a predominantly identical nucleotide sequence to a region of the unmodified sequence in which mutation is desired, other than changes at the nucleotide residues appropriate to bring about the desired alteration. Subsequent use of this primer in a PCR amplification reaction will thus introduce the desired changes to the nucleotide sequence of the PCR product. The desired site for site directed mutagenesis is typically within the coding sequence of the gene, and thus the PCR product may be a truncated form of the target polynucleotide. A full length modified polynucleotide of the invention may be generated by combining the amplification product with other polynucleotides that have unmodified or modified sequences (generated by any technique). Combination may be performed by any technique known in the art, e.g. ligation. This technique may also be useful where for example silent codon changes are required to optimise codon preferences for a particular host cell in which the polynucleotide sequences are being expressed. Other sequence changes may be desired in order to introduce restriction enzyme recognition sites, or to further alter or modify the property or function of the polypeptide encoded by the polynucleotide.

The modified polynucleotide generated may be tested for the desired sequence by its sequencing. This may be performed, for example, by bringing a sample containing the putative modified polynucleotide, as target, into contact with a probe comprising a polynucleotide or primer of the invention under hybridizing conditions and determining the sequence by, for example the Sanger dideoxy chain termination method (see Sambrook et al, 2001, or 1989 ref as above).

Such a method generally comprises elongating, in the presence of suitable reagents, the primer by synthesis of a strand complementary to the target polynucleotide and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Polynucleotides of the invention can be incorporated into any vector available in the art. A vector of the invention consists essentially of a polynucleotide of the invention, therefore. Usually the vector will be a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

Preferably, a polynucleotide of the invention in a vector is operably linked to a control sequence which is capable of providing for the expression of the coding sequence by the host cell, i.e. the vector is an expression vector. Such expression vectors can be used to express polypeptides of the invention.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable host cell as described above to provide for expression of a polypeptide or polypeptide fragment of the invention. Thus, in a further aspect the invention provides a process for preparing polypeptide according to the invention, which process comprises cultivating a host cell transformed or transfected with an expression vector as described above under conditions to provide for expression of the polypeptide, and recovering the expressed peptide.

The vectors may be for example, plasmid, virus or phage vectors provided with an origin of replication, optionally a promoter for the expression of the said polynucleotide and optionally a regulator of the promoter. The vectors may contain one or more selectable marker genes, for example, an ampicillin resistance gene in the case of a bacterial plasmid, a neomycin resistance gene for a mammalian vector, or a kanomycin resistance gene for a plant vector. Vectors may be used in vitro, for example for the production of RNA or used to transfect or transform a host cell. The vector may also be adapted to be used in vitro, for example in a method of gene therapy.

A further embodiment of the invention provides host cells transformed or transfected with the vectors for the replication and expression of polynucleotides of the invention. The cells will be chosen to be compatible with the said vector and may for example be bacterial, yeast, plant, insect, or mammalian.

Expression vectors of the invention may be introduced into host cells using conventional techniques including calcium phosphate precipitation, DEAE-dextran transfection, electroporation, particle bombardment or Agrobacterium tumefaciens-mediated techniques. Expression from the host cell may be transient. Stable host cell transformation may be achieved by integration of a polynucleotide of the invention, or a fragment thereof, into a genome of the host cell. Typically the transformed genome is nuclear, although transformation of other genomes may be desired, for example the mitochondrial genome of eukaryotic cells, or a plastidic genome of plant cells. Alternatively, stable transformation may be achieved using replicable autonomous vectors. The expression vector may contain a selectable marker and/or such a selectable marker may be co-transfected with the expression vector and stable transfected cells may be selected.

Suitable cells include cells in which the abovementioned vectors may be expressed. These include microbial cells such as bacteria such as E. coli, mammalian cells such as CHO cells, COS7 cells, P388 cells, HepG2 cells, KB cells, EL4 cells or Hela cells, insect cells, yeast such as Saccharomyces or plant cells, typically of crop plants such as wheat, maize or oil-seed rape. Baculovirus or vaccinia expression systems may be used.

Cell culture will take place under standard conditions. Commercially available cultural media for cell culture are widely available and can be used in accordance with manufacturer's instructions.

Peptides of the invention expressed in host cells may be recovered by any technique known in the art. This may lead to isolation and purification of the polypeptide; Typically an isolated or purified polypeptide will account for at least 10% to 100% dry mass of the polypeptide present in the sample, more preferably at least 40% or 50%, even more preferably at least 60% or 70% yet more preferably at least 80%, 90%, 95%. Most preferably a purified polypeptide will account for at least 99% by dry mass of the polypeptide present in a sample.

The present invention may also include taking a selected naturally occurring polypeptide such as human calcitonin, analysing the amino acid sequence to assess the tendency of the polypeptide to aggregate, designing a modified polypeptide based on the human calcitonin sequence which would have a reduced tendency to aggregate compared to the wild type form and producing such a modified polypeptide.

Any of the peptides, polynucleotides, vectors, cells, discussed above in any form or in association with any other agent discussed above is included in the term ‘agent’ below. An effective non-toxic amount of such a agent may be given to a human or non-human patient in need thereof. The condition of a patient suffering from a disease can therefore be improved by administration of such an agent. The agent may be administered prophylactically to an individual who does not have a disease in order to prevent the individual developing the disease.

Thus the invention provides the agent for use in a method of treating the human or animal body by therapy. The invention provides the use of the agent in the manufacture of a medicament for treating the disease. Thus the invention provides a method of treating an individual comprising administering the agent to the individual.

The agent is typically administered by any standard technique used for administration, such as by injection or intranasal spray.

Typically after the initial administration of the agent, the same or a different agent of the invention can be given. In one embodiment the subject is given 1, 2, 3 or more separate administrations, each of which is separated by at least 6, 12 hours, 1 day, 2, days, 7 days, 14 days, 1 month or more.

The agent may be in the form of a pharmaceutical composition which comprises the agent and a pharmaceutically acceptable carrier or diluent. Suitable carriers and diluents include isotonic saline solutions, for example phosphate-buffered saline. Typically the composition is formulated for parenteral, intravenous, intramuscular, subcutaneous, transdermal, intradermal, oral, intranasal, intravaginal, or intrarectal administration.

The dose of administration may be determined according to various parameters, especially according to the substance used; the age, weight and condition of the patient to be treated; the route of administration; and the required regimen. A physician will be able to determine the required route of administration and dosage for any particular patient. A suitable dose may however be from 10 μg to 10 g, for example from 100 μg to 1 g of the agent. These values may represent the total amount administered in the complete treatment regimen or may represent each separate administration in the regimen.

In the case of agents which are polynucleotides transfection agents may also be administered to enhance the uptake of the polynucleotides by cells. Examples of suitable transfection agents include cationic agents (for example calcium phosphate and DEAE-dextran) and lipofectants (for example lipofectam™ and transfectam™)

When the agent is a polynucleotide which is in the form of a viral vector the amount of virus administered is in the range of from 10⁴ to 10¹² pfu, preferably from 10⁷ to 10¹⁰ pfu (for example for adenoviral vectors), more preferably about 10⁸ pfu for herpes viral vectors. A pox virus vector may also be used (e.g. vaccinia virus), typically at any of the above dosages. When injected, typically 1-2 ml of virus in a pharmaceutically acceptable suitable carrier or diluent is administered. In a preferred aspect of the present invention, the peptide is modified human calcitonin. The modified calcitonin is provided for use in the treatment of Paget's disease, aspects of hypercalcaemia or osteoporosis, and in particular hypercalcaemic crisis due to: tumoral osteolysis secondary to breast, lung, kidney and other malignancies, osteolysis induced by myeloma, primary hyperparathyroidism, Paget's disease of bone (osteitis deformans), particularly in cases with bone pain, neurological complications, increased bone turnover reflected in elevated alkaline phosphatase and hydroxyproline secretion, progressive extension of bone lesions, incomplete or repeated fractures; pain associated with advanced metastatic bone cancer or other cancer; other forms of pain resistant to conventional treatments; short term use in post menopausal osteoporosis and other treatments related to any other therapeutic or physiological activity described for human, salmon or pig calcitonin or any other therapeutic form of calcitonin.

The invention is now described in more detail by reference to the following example:

Naturally occurring Calcitonin sequences are as follows:

| | | |
|---|---|---|
| CGNLSTCMLGTYTQDFNKFHTFPQTAIGVGAP | Human | SEQ ID |
| | | NO 1 |
| CGNLSTCMLGTYTQDLNKFHTFPQTSIGVEAP | Mouse | SEQ ID |
| | | NO 2 |
| CGNLSTCMLGTYTQDLNKFHTFPQTSIGVGAP | Rat | SEQ ID |
| | | NO 3 |
| CSNLSTCVLSAYWKDLNNYHRFSGMGFGPETP | Bovine | SEQ ID |
| | | NO 4 |
| CSNLSTCVLGTYSKDLNNFHTFSGIGFGAETP | Dog | SEQ ID |
| | | NO 5 |
| CSNLSTCVLSAYWRNLNNFHRFSGMGFGPETP | Pig | SEQ ID |
| | | NO 6 |
| CSNLSTCVLSAYWKDLNNYHRYSGMGFGPETP | Sheep | SEQ ID |
| | | NO 7 |
| CSNLSTCMLGKLSQDLHKLQTFPRTNTGAGVP | Salmon-3 | SEQ ID |
| | | NO 8 |
| CSNLSTCVLGKLSQDLHKLQTFPRTNTGAGVP | Salmon-2 | SEQ ID |
| | | NO 9 |
| CSNLSTCVLGKLSQELHKLQTYPRTNTGSGTP | Salmon-1 | SEQ ID |
| | | NO 10 |
| CSNLSTCVLGKLSQELHKLQTYPRTDVGAGTP | Eel | SEQ ID |
| | | NO 11 |
| CASLSTCVLGKLSQELHKLQTYPRTDVGAGTP | Chicken | SEQ ID |
| | | NO 12 |

The considerations for engineering new variants were as follows. Firstly, only those residues that constitute a polymorphism between species were selected for modification. In this way, at least some of the physiological function of calcitonin could be retained. This is particularly important in the cyclical N-terminus domain in which cysteins 1 and 7 form a disulfide bridge, which is considered specific for receptor activation.

The number of changes was kept to a minimum. The higher the sequence identity compared to the human form the lower the probability of resistance generation by the patient.

Peptide sequences were designed as follows:

| | |
|---|---|
| Agadir | GOR-4 |

| | | | | |
|---|---|---|---|---|
| CGNLSTCMLGTYTQDFNKFHTFPQTAIGVGAP | Human | | 0.43% | 5.5% |
| CSNLSTCVLGKLSQELHKLQTYPRTNTGSGTP | Salmon-1 | | 2.03% | 18.8% |
| CGNLSTCMLGKLTQELNKLHTFPQTAIGVGAP | 5ch | SEQ ID NO 13 | 3.10% | 19.2% |
| CGNLSTCMLGKLTQELRKLHTFPQTAIGVGAP | 6chR | SEQ ID NO 14 | 6.17% | 22.0% |
| CGNLSTCMLFTYTQDFRKFHTYPQTAIGVGAP | 1chR | SEQ ID NO 15 | 0.88% | 8.4% |
| CGNLSTCMLGKLTQELLKLHTFPQTAIGVGAP | 6chL | SEQ ID NO 16 | 6.54% | 23.7% |
| CGNLSTCMLGKLTQELLKLLTFPQTAIGVGAP | 7ch | SEQ ID NO 17 | 16.57% | 28.7% |
| CGNLSTCMLGKLTQELLKLLTFPRTAIGVGAP | 8ch | SEQ ID NO 18 | 17.77% | 28.9% |

Agadir predictions are made under Standard Conditions (100 mM ion strength, pH 7.0) and arnidated C-terminus. GOR-4 predictions are made using Standard Parameters.

The variant 6 chR was selected for further investigation. This variant constitutes a starting point with only 6 changes with respect to the human sequence (>81% sequence identity). In later stages the number of changes is reduced to 5 changes >84% identity to make a product even more similar to the human form without its high aggregation tendency. 6 chR is selected instead 6 chL to prevent an excessive concentration of hydrophobic residues and also to provide additional charges that could improve peptide solubility (basic residues would also do this at acidic Ph values). A Basic residue was chosen given that the salmon calcitonin has at that position a Histidine residue (also basic).

Peptides were synthesised with a cyclic N-terminus and C-terminus amidation.

| | |
|---|---|
| CGNLSTCMLGTYTQDFNKFHTFPQTAIGVGAP | Human |
| CGNLSTCMLGKLTQELRKLHTFPQTAIGVGAP | 6chR |

Aggregation Studies

Aggregation analysis was carried out as reported previously (Arvinte, T. et al., 1993, J. Biol. Chem, 268, 6415-6422) using PBS 5 mM sodium phosphate, 145 mM NaCl, pH 7.2. (sodium phosphate saline buffer) pH 7.2 and adding a minor amount of sodium azide to prevent bacterial growth (0.1%). Samples were always incubated at 37° C.

Aggregation was monitored by measuring turbidity at 340 nm. Different concentrations of peptide were tested. Samples were processed (filtered or not) as indicated in table 1, below.

TABLE 1
Aggregation times for Human and 6CHR variant at different peptide
concentrations^{a,b}

| Concentration | Human | 6CHR |
|---|---|---|
| 10 mM (˜35 mg/mL) | 4 hours | 18 days |
| unfiltered | | |
| 10 mg/mL unfiltered | ˜30 hours | (>10 months)^{c} |
| 10 mg/mL filtered 0.5 μm | ˜26 days | (>10 months)^{c} |
| 1 mg/mL filtered 0.5 μm | ˜45 days | (>10 months)^{c} |

^{a}Times of aggregation represent half times followed by turbidity at 340 nm. When aggregation kinetics exceeded 10 days time of aggregation was determined when development of turbidity appeared. Samples were centrifuged at 13000 g before incubation.
^{b}In all the cases incubation was at 37° C. and buffer was 10 mM sodium phosphate, 150 mM NaCl, pH 7.2. Sodium azide 0.01% was added to prevent bacterial growth.
^{c}(>10 months) means that no aggregation was detected at the end of the experiment

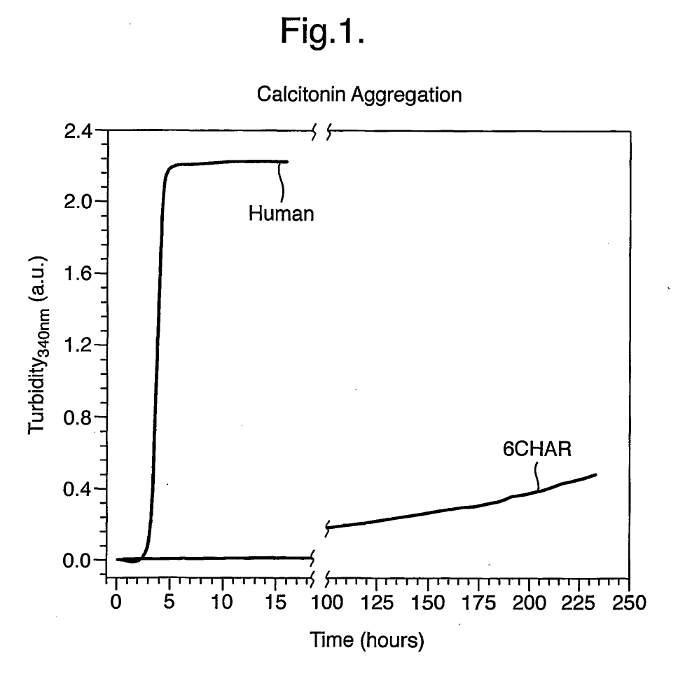

FIG. 1 shows the aggregation behaviour of human calcitonin and the 6 chR variant when dissolved at a peptide concentration of 35 mg/mL and solutions were not filtered. Clearly the variant aggregates much more slowly than the human form. At concentrations of 10 mg/mL or below 6 CHR in both unfiltered and filtered solutions showed no detectable aggregation over >10 months, at the end of the experiment. Table 1 reflects the aggregation time of human and 6 chR calcitonin under different solution conditions. Filtering solutions slowed the aggregation kinetics for the human variant, however in all the cases the 6 chR variant is less prone to aggregate. Samples exhibiting turbidity were checked by electron microscopy for amyloid fibrils, and in all the cases they exhibited similar fibrillar structures only after turbidity was present in the samples. We can say that the time required for the 6 chR variant to aggregate is at least two orders of magnitude larger than that one required for the human peptide.

Structural Studies

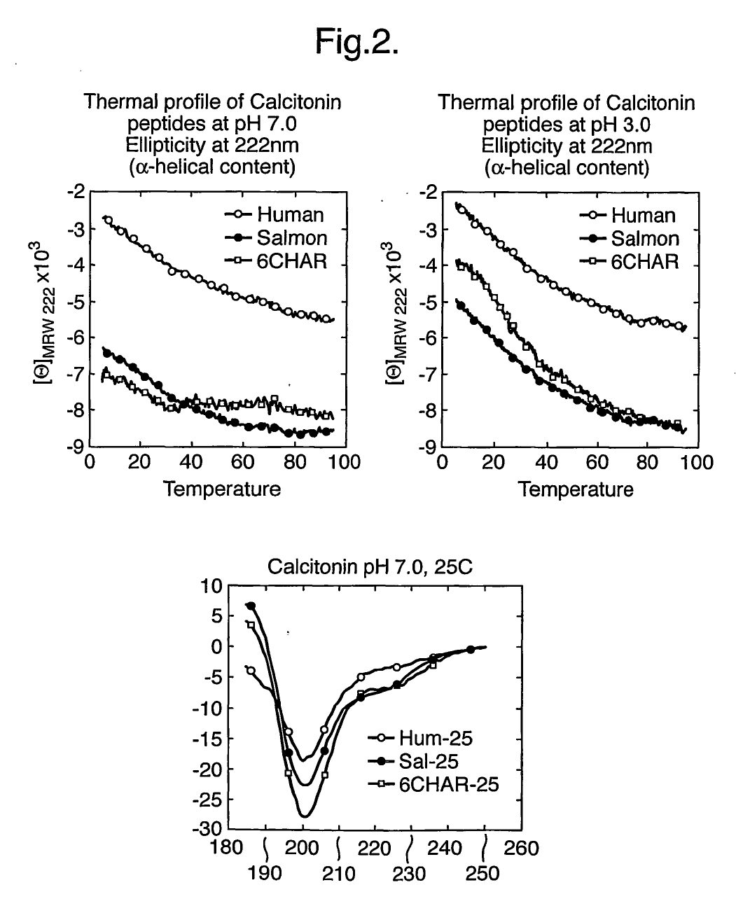

Circular dichroism and Nuclear magnetic resonance were used to characterise the structural properties of human (hCT), salmon (sCT) and 6 chR (6 chR) calcitonin. Peptide concentration was estimated by amino acid analysis. FIG. 2 shows that sCT (22.5%) has exhibits a higher helical content than hCT (11.3%) at neutral pH in agreement with previous observations, as well as at low pH values. When compared with hCT, 6 chR (21.1%) exhibits also an enhanced helical content at both neutral and low pH in agreement with the predictions made. This enhanced helical content is also detected by chemical shift analysis using NMR (data not shown). Therefore these tests suggest a success in the designing procedure both in terms of stabilisation of helical local interactions and stabilisation of the 6 chR peptide in terms of aggregation.

Physiological Activity

The next step in the experimental strategy was to establish whether or not the engineered peptide besides being much less prone to aggregation than the wild type human form showed a calcitonin-like activity and therefore could be used as a therapeutic agent. Activity tests may be carried out in suitable animal models using, for example chickens or mice. Activity can also be monitored in vitro, for example in cellular cultures. LLC-PKI kidney cells may be used, for which calcitonin has been reported to increase alkaline phosphatase activity and cAMP levels Wohlwend A et al (1985) Biochem Biophys Commen 131 537-542, Miyamoto K. I. et al (1998) Jpn. J. Pharmacol 76.193-198. Activity tests described below were carried on cellular systems that allow a better control of the experimental conditions in order to compare the activities exhibited by different calcitonin forms. We used a combination of two previously described systems with some modifications (Zimmerman, U. et al, 1997, J. Endocrinol. 155, 423-431; Miyamoto, K. I. et al, 1998, Jpn. J. Pharmacol 76, 193-198). Human ductal carcinoma T47D (HTB 133) cells were used for the assays.

Cell Culture

T47D (HTB 133) cells were purchased from the American Type Culture Collection (ATCC) and grown in RPMI-1640 modified medium (ATCC) containing 2 mM L-glutamine, 10 mM HEPES, 1 mM sodium pyruvate, 4.5 g/L glucose and 1.5 g/L sodium bicarbonate. This medium was supplemented with 10% heat-inactivated fetal calf serum, 25 μg/mL gentamycin and 100 nM dexamethasone, this last to enhance the expression of calcitonin receptors. Cells were grown in a humidified atmosphere of 95% air-5% CO₂ at 37° C.

Determination of Calcitonin Activity by Measurement of Cyclic AMP Intracellular Accumulation

Cyclic AMP (cAMP) accumulation was used to measure the degree of stimulation upon incubation in the presence of different calcitonin peptides. Typically cells were grown in multi-well plates until confluency was reached and then washed with pre-heated DPBS-glucose (Gibco, Invitrogen) to remove any traces of fetal serum. Sample solutions were prepared by dissolving different amounts of hCT, sCT and 6 chR (6 CT) in DPBS-glucose supplemented with 0.1% BSA and 1 mM IBMX (3-isobuthyl-1-methylxanthine). After being washed, cells were incubated for 15 min at 37° C. in the presence of the peptide solutions. Medium was then removed by aspiration and cells were rapidly frozen on dry-ice and then kept at −80° C. until cAMP measurements were carried out.

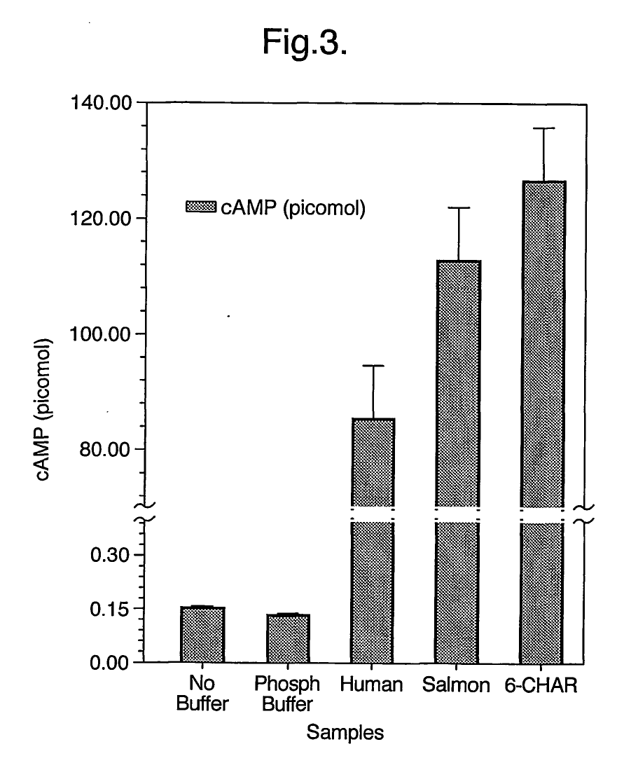

Cyclic AMP content of different samples was measured using a commercial AMP enzyme immunoassay (EIA) system (Biotrak, Amersham-Biotech) according to manufacturer's specifications. Different dilutions of cellular extracts were measured in order to cover the whole range of stimulation caused by calcitonin incubation. The results are shown in FIG. 3. Saturation concentrations (1·10⁻⁶ M) of the three peptides (hCT, sCT & 6chR) were used to establish the maximum capacity of stimulation for each one of the different sequences. In all cases the stimulation observed is close to three orders of magnitude compared with the basal levels. All three peptides show clear stimulation effects confirming the calcitonin-like activity of the engineered 6 chR (6 CT) peptide. Moreover the stimulation effect obtained by incubation with 6 chR is clearly higher than that of the hCT and even the sCT peptide (the major current therapeutic form).

All these results clearly demonstrate that the design strategy followed is valid and extremely powerful to devise new forms of calcitonin that keeping a high sequence identity with the human form show a dramatic reduction in terms of aggregation and keep or even improve their activity, which makes these ‘humanised’ designs suitable candidates to use as therapeutic agents complementing or replacing current therapeutic forms of calcitonin. Moreover these studies open new possibilities for the design of improved forms of other therapeutic or bio-active peptides, such as CGRP-1, CORP-2, amylin or adrenomedullin (part of the calcitonin family) or even sequences not related to those, given the generality of the approach employed.

Other Bioactive Peptides Related With Calcitonin (Cysteines that Participate in the Conserved Intra-Chain Disuifide Bridge Appear Underlined)

| Adrenomedullin human | SEQ ID NO 19 |
|---|---|
| YRQSMNNFQGLRSFGCRFGTCTVQKLAHQIYQFTDKDKDNVAPRSKISPQGY | |
| CGRP-1 human | SEQ ID NO 20 |
| ACDTATCVTHRLAGLLSRSGGVVKNNFVPTNVGSKAF | |
| CGRP-2 human | SEQ ID NO 21 |
| ACNTATCVTHRLAGLLSRSGGMVKSNFVPTNVGSKAF | |
| IAPP (Amylin) human | SEQ ID NO 22 |

KCNTATCATQRLANFLVHSSNNFGAILSSTNVGSNTY

AM (adrenomedullin) is a potent hypotensive and vasodilatator agent. Numerous actions have been reported most related to the physiologic control of fluid and electrolyte homeostasis. In the kidney, AM is diuretic and natriuretic, and AM inhibit aldosterone secretion by direct adrenal actions. In pituitary gland, both peptides at physiologically relevant doses inhibit basal acth secretion. AM appear to act in brain and pituitary gland to facilitate the loss of plasma volume, actions which complement their hypotensive effects in blood vessels

CGRP (calcitonin gene-related peptide) induces vasodilatation. It dilates a variety of vessels including the coronary, cerebral and systemic vasculature. Its abundance in the CNS also points toward a neurotransmitter or neuromoduiator role. It also elevates platelet cAMP. There are 3 CGRP isoforms; 1 (Calcitonin), 2 (CGRP-1) and 3 (CGRP-2 ); that are produced by alternative splicing of the same gene. Belongs to the calcitonin family.

IAPP (amylin) selectively inhibits insulin-stimulated glucose utilization and glycogen deposition in muscle, while not affecting adipocyte glucose metabolism. IAPP is the peptide subunit of amyloid found in pancreatic islets of type 2 diabetic patients and in insulinomas. Belongs to the calcitonin family.

A similar design approach to that described above for calcitonin can be undertaken with these four and other similar peptides to generate improved varieties that present a reduced aggregation propensity.

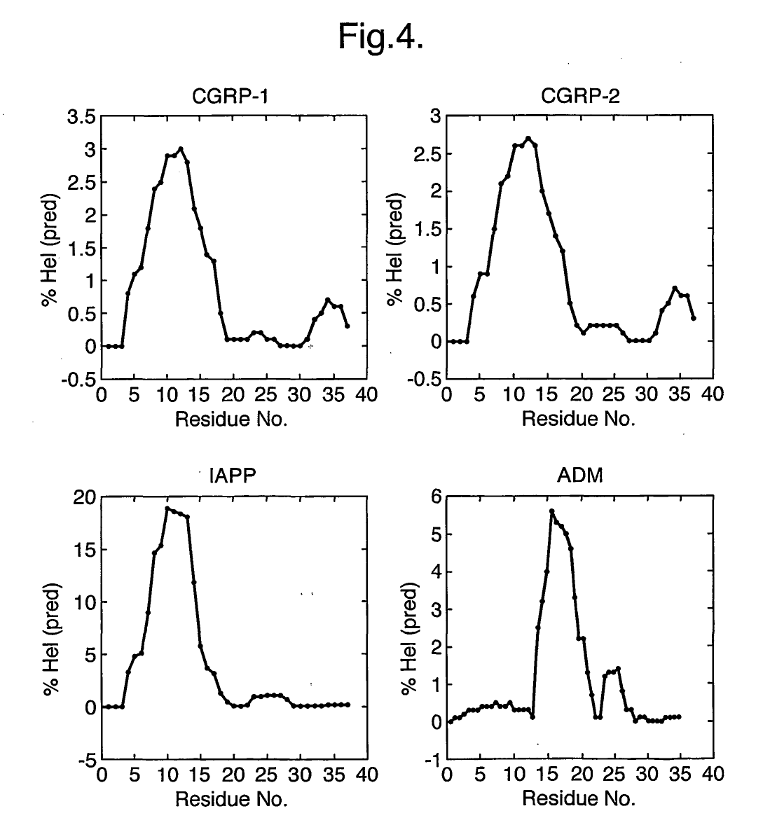

FIG. 4 shows helical propensity prediction profiles for different calcitonin-related peptides. In all cases a central region with higher helical propensity can be located. This region is susceptible to be modified to enhance α-helical interactions as described previously with calcitonin. These designable regions are residues 20-32 and 35-41 for adrenomedullin (ADM), and 8-20 for CGRP-1, CGRP-2 and IAPP (amylin) (see FIG. 4). The modified peptides will preferably exclude any modification in or before the conserved intra-chain disulfide bridge, which seems to be important in their physiological activity. Modifications will preferably be made in residues with higher propensity to form helical contacts (see FIG. 4 and above) and preferably will avoid modifications in the C-terminus part of the peptides such as the terminal 5 or terminal 10 amino acids. Thus preferably modifications may be made in one or more residues of 20-32 and/or 35-41 of adrenomedullin, one or more residues of 8-20 for CGRP-1, CGRP-2 and IAPP (amylin). Engineered substitutions would be preferably in those amino acids that constitute polymorphisms among species and would preferably attempt to stabilise α-helical interactions. Additionally substitutions would substitute hydrophobic amino acids for other polar or less hydrophobic. Introduction of charges to generate repulsion and stabilise the soluble form of the peptides would also be performed, with preference for basic residues.

1. Modified human calcitonin, comprising a peptide having at least 70% identity to SEQ ID No 1 and being modified such that the tendency of the modified peptide to aggregate is reduced compared to unmodified human calcitonin.

2. Modified calcitonin according to claim 1, wherein the peptide differs from SEQ ID No 1 at no more than 6 amino acid residues.

3. Modified calcitonin according to claim 2, wherein the peptide differs from SEQ ID No 1 at no more than 5 amino acid residues.

4. Modified calcitonin according to any one of the preceding claims, wherein the peptide is unmodified compared to human calcitonin within the 5 N-terminal amino acids.

5. Modified calcitonin according to any one of the preceding claims, wherein the peptide is unmodified compared to human calcitonin within the 10 N-terminal amino acids.

6. Modified calcitonin according to any one of the preceding claims, wherein the peptide is unmodified compared to human calcitonin within the 5 C-terminal amino acids.

7. Modified calcitonin according to any one of the preceding claims, wherein the peptide is unmodified compared to human calcitonin within the 10 C-terminal amino acids.

8. Modified calcitonin according to any one of claims 1 to 6 in which the amino acids for substitution in SEQ ID NO: 1 are selected from 11, 12, 15, 16, 17, 19, 20 and 24.

9. A polypeptide according to claim 8 wherein two or more amino acids at positions 11, 12, 15, 16, 17, 19, 20 and 24 are substituted compared to wild type human calcitonin.

10. Modified calcitonin according to any one of the preceding claims, wherein the peptide is selected from
CGNLSTCMLGKLTQELNKLHTFPQTAIGVGAP,
CGNLSTCMLGKLTQELRKLHTFPQTAIGVGAP,
CGNLSTCMLGKLTQELLKLHTFPQTAIGVGAP,
CGNLSTCMLGKLTQELLKLLTFPQTAIGVGAP, or
CGNLSTCMLGKLTQELLKLLTFPRTAIGVGAP.

11. A pharmaceutical composition comprising a modified peptide according to any one of the preceding claims and a pharmaceutically acceptable carrier.

12. A pharmaceutical composition or a modified peptide according to any one of the preceding claims for use in the treatment of paget's disease, hypercalcaemia or osteoporosis.
